(19) United States
(12) Patent Application Publication
(10) Pub. No.: US 2012/0316419 A1
(43) Pub. Date: Dec. 13, 2012

(54) MULTIMODAL CATHETER

(76) Inventor: Eric Chevalier, Pully (CH)

(21) Appl. No.: 13/580,116

(22) PCT Filed: Feb. 18, 2011
(86) PCT No.: PCT/IB11/50686

§ 371 Date: Aug. 20, 2012

Related U.S. Application Data
(60) Provisional application No. 61/305,605, filed on Feb. 18, 2010.

(51) Int. Cl. A61B 8/06; A61B 5/04; A61B 8/12; A61B 5/027

(52) U.S. Cl. 600/381; 600/505; 600/468

(57) ABSTRACT

Catheter based system for providing functional and morphological characterization of arteries, comprising a catheter (1) configured for insertion in an artery (3), and a sensor system (5) for mapping hemodynamic parameters mounted on the catheter (1), the sensor system comprising at least two anemometric probes (7, 8a, 8b, 9a, 9b, 19, 20, 21, 22) spatially arranged in a deployed position and configured to measure flow velocity components (Vx, Vr) in at least two different positions spaced apart in a radial direction R of such that a possible restriction of the artery due for example to a stenosis, plaque, or other local deformation (3a) of the artery is measurable.

### FIELD OF THE INVENTION

This invention relates generally to catheter-based intravascular sensing methods and devices, including blood flow sensing, morphological artery sensing and biochemical arterial sensing.

### BACKGROUND

For many years, exploration and treatment of various organs or vessels has been possible using catheter-based diagnostic and treatment systems. Such catheters are introduced through a vessel leading to the cavity of the organ to be explored or treated or alternatively may be introduced directly through an incision made in the wall of the organ. In this manner, the patient avoids the trauma and extended post operative recuperation time typically associated with open surgery. Intravascular (i.e. cardiovascular, neurovascular . . . ) catheter-based diagnostic and treatment procedures have become common clinical practices.

During the past decade, the physiological assessment of coronary artery disease (CAD) has become increasingly important in both clinical and research applications. Angiography alone, the current gold standard, cannot fully characterize the clinical significance of coronary stenosis. This well-recognized limitation has been documented repeatedly by intravascular ultrasound imaging and ischemia stress testing. Coronary angiography produces a silhouette image and cannot identify intraluminal detail or provide the angiographer with information about the characteristics of the vessel wall. Furthermore, accurate identification of both normal and diseased vessel segments is complicated by diffuse disease as well as by angiographic artefacts of contrast streaming, image foreshortening, and calcification. Bifurcation or ostial lesion locations may be obscured by overlapping branch segments. Even with numerous angiographic angulations to reveal the lesion in its best view, the physiological significance of a coronary stenosis, especially for an intermediately severe luminal narrowing (approximately 40% to 70% diameter narrowing), cannot be accurately determined. This fact is demonstrated by the clinical uncertainty of the angiographer, with confirmatory ischemic stress testing frequently needed in patients with such “moderate” CAD. Like stress testing, measurements of coronary pressure and flow provide information complementary to the anatomic characterization of coronary disease obtained by both angiographic and intravascular ultrasound examinations. Such physiological data acquired during the angiographic procedure can facilitate timely and more objective decision-making about therapy. Thus, the rationale for using coronary physiological measurement is to overcome the limitations of coronary angiography and provide the angiographer with an objective indicator of clinically relevant lesion significance. (M. J. Kern & al. Circulation 2006; 114; 1321-1341)

An important physiological index is the fractional flow reserve: it measures the maximum achievable myocardial blood flow in the presence of a coronary artery stenosis as a percentage of the maximum blood flow in the hypothetical case of a completely normal artery. Such index can be estimated by a commercially available pressure sensing wire, the RADI PressureWire. It is also known to integrate a small temperature sensitive resistor used as a hot film anemometer. (A. Van Der Horst, MSc Thesis, Eindhoven, 2007, R. Van der Sligte, MSc Thesis, Eindhoven, 2009). The principle of anemometry applied for Hemodynamics was studied by Kaulsen & al., Med. & Biol. Eng. & Comput., 1982, 20, 625-627. However, there remain important limitations for an accurate clinical diagnosis. Although sensor guidewire technology provides local measurements at single points along the artery and may be combined with angiography, an accurate set of stenosis characterizations (geometry/topography) are not possible or not easily or reliably achieved. Also, advances in the understanding of atheromatous coronary disease have focused on the composition of the plaque rather than the degree of stenosis as the major pathophysiologic determinant of the acute manifestations of the disease (plaque disruption . . . ).

In recent years, cardiovascular research has sought potential strategies for detecting high risk plaques before their disruption. These potentially powerful techniques are aimed at identification of populations at risk and plaque monitoring and might eventually guide targeted therapy.

Various techniques for physiologic circulation in arteries and detection of vulnerable plaque have been proposed, as briefly discussed below.

In US2009270695, there is described a method of measurement of sound speed both intravascularly and non-invasively with the acoustic transducer(s) mounted non-invasively on opposite side of a vessel or artery or on an intravascular catheter. The intravascular catheter incorporates a sensor to measure whole blood sound velocity, attenuation, backscatter amplitude, and blood flow velocity and also incorporates existing technologies for multiple physiologic measurements of whole blood. Pulse wave velocity and wave intensity are derived mathematically for purposes of estimating degree of local vascular tone.

Catheters that include sensors to measure blood flow are well known per se. U.S. Pat. No. 5,280,786 describes a fiberoptic blood pressure and oxygenation sensor deployed on a catheter placed transcutaneously into a vessel. A sensing tip of the catheter includes a pressure-sensing element and an oxygen saturation-measuring element.

It is also known that blood flow, or velocity can be measured by Doppler ultrasound methods. For Example, U.S. Pat. No. 6,616,611 describes a Doppler ultrasound method and apparatus for monitoring blood flow describes a pulse Doppler ultrasound system and associated methods are described for monitoring blood flow. It has been contemplated that Doppler ultrasound sensors can be placed internally. U.S. Pat. No. 6,704,590 describes a doppler guiding catheter using a piezoelectric sensor or an optical sensor at the tip to show turbulence through a time domain or frequency domain presentation of velocity. The sensor readings can be used to modulate an audible waveform to indicate turbulence. Detecting changes in a blood flow turbulence level is used to assist guiding of the distal end of the flexible shaft.

Intravascular ultrasound (IVUS) is a medical imaging technology. It uses a specially designed catheter that includes an ultrasound transducer. The catheter is inserted into the vascular system of a patient and moved to an artery or vein of interest. It allows the doctor to obtain an image of the endothelium (inner wall) of vessels, and structures within the vessel walls even through intervening blood. IVUS is used in coronary arteries of the heart to locate, identify and characterize atherosclerotic plaques in patients. It can be used both to determine the plaque volume in the piping wall and also the degree of stenosis (narrowing) of the vessels.

Optical coherence tomography (OCT) is an emerging technology that also provides structural information similar to IVUS. OCT also uses a catheter that is moved through the vessels to regions of interest. An optical signal is emitted from the catheter head and the returning signal is analyzed for phase or coherence in a Michelson interferometer. OCT has potential advantages over IVUS. Generally, OCT provides the opportunity for much higher spatial resolution, but the optical signals have limited penetration through blood and attenuate very quickly when propagating through the walls of the vessels. An objective to using systems based on OCT and IVUS structural imaging technologies is the early identification of vulnerable plaques since disruption or rupture of atherosclerotic plaques appears to be the major cause of heart attacks and strokes. After the plaques rupture, local obstructive thromboses form within the vessels. Both venous and arterial thrombosis can occur. A coronary thrombus often initially forms at the site of rupture of a vulnerable plaque; i.e. at the location of a plaque with a lipid-rich core and a thin fibrous cap (thin-cap fibroatheroma or TCFA).

Another class of intravascular analysis systems directed to the diagnosis and analysis of atherosclerosis uses chemical analysis modalities. These approaches generally rely on optical analysis including near infrared (NIR), Raman, and fluorescence spectral analysis. Probably the most common and well developed of these chemical analysis modalities is NIR analysis of the vessel walls (J. Wang & al., J. Am. Coll. Cardiol. 2002; 39; 1305-1313).

Similar to OCT, NIR analysis utilizes an intravascular optical catheter, in a typical application, the catheter is driven by a pullback and rotation unit that simultaneously rotates the catheter head around its longitudinal axis while withdrawing the catheter head through the region of the vessel of interest. During this pullback operation, the spectral response of the inner vessel walls is acquired in a raster scan operation. This provides a spatially-resolved spectroscopic analysis of the region of interest. By determining the spectroscopic response of vessel walls, the chemical constituents of those vessel walls can be determined by application of chemometric analysis. In this way, potentially vulnerable plaques are identified so that, for example, stents can be deployed in order reduce the risk of myocardial infarction. In NIR analysis, the blood flow does not necessarily have to be occluded during the analysis. The judicious selection of the wavelengths of the optical signals allows adequate penetration through intervening blood to the vessels walls and back to the catheter head. In Raman spectral analysis, the inner walls of the vessel are illuminated by a narrow band, such as laser, signal. The Raman spectral response is then detected. This response is generated by the inelastic collisions between photons and the chemical constituents in the vessel walls. This similarly produces chemical information for the vessel walls. Problems associated with Raman analysis are, however, that the Raman process is a very weak and requires the use of high power optical signals in order to generate an adequate Raman response. Fluorescence has some advantages in that the fluorescence response is sometimes much larger than the Raman response. Generally, however, fluorescence analysis does not yield as much information as Raman or NIR analysis.

In an effort to obtain valuable information from both the chemical and structural analysis modalities, hybrid IVUS/optical catheters have been proposed. In U.S. Pat. No. 6,949,072, a “device for vulnerable plaque detection” is described. The device is directed to an intravascular probe that includes optical waveguides and ports for the near infrared analysis of the vessels walls while simultaneously including an ultrasound transducer in the probe in order to enable IVUS analysis of the vessel walls.

In PCT publication WO2009124242, there is disclosed a multimodal intravascular analysis based in chemical analysis and structural analysis of vessel walls. The proposed device integrates ultrasound or optical techniques and NIR or Raman.

It has been said that “The ideal technique would provide morphological, mechanical and biochemical information, however, despite several imaging techniques are currently under development, none of them provides alone such all-embracing assessment. Thus the combination of several modalities will be of importance in the future to ensure a high sensitivity and specificity in detecting vulnerable plaques.”(Ref. Pierfrancesco Agostoni, Johannes A. Schaar, Patrick W. Serruys, Kardiovaskuläre Medizin 2004; 7:349.358).

### SUMMARY OF THE INVENTION

An object of the invention is to provide a catheter based system for providing functional and morphological characterization of arteries that is accurate, reliable and cost-effective.

It is advantageous to provide a catheter based system for mapping hemodynamic parameters that is accurate, reliable and cost effective.

It is advantageous to provide a catheter based system for providing functional and morphological characterization of arteries that is minimally invasive.

It is advantageous to provide a catheter based system for providing functional and morphological characterization of arteries that provides extensive information useful for diagnosing various arterial conditions or diseases, including an accurate representation of plaques and stenosis.

It is advantageous to provide a multimodal catheter with accurate and reliable hemodynamic sensing.

It is advantageous to provide an accurate and reliable multimodal catheter incorporating hemodynamic sensing, and/or biochemical sensing and/or morphological artery sensing.

The present invention proposes a catheter based system for providing functional and morphological characterization of arteries, comprising a catheter configured for insertion in an artery, and a sensor system mounted on the catheter, the sensor system comprising at least two anemometric probes for mapping hemodynamic parameters spatially arranged in a deployed position and configured to measure flow velocity components in at least two different positions spaced apart in a direction orthogonal to the axial direction (i.e. a radial direction) of the catheter essentially in a same plane or within a short axial distance or zone of the artery.

The anemometric probes may advantageously comprise hot thin-film probes. Some or all of the sensor probes may be mounted on a deployable structure configured to expand in at least a radial direction from a retracted position allowing insertion of the catheter in an artery, to the deployed position when the measurement process for characterization of the arteries is implemented. Instead of hot thin-film probes, the anemometric probes may also be in the form of hot wire probes or anemometric probes of other configurations. The use of hot thin-film probes is however particularly advantageous in view of the easy and flexible forming thereof in a desired configuration on a deployable structure, and the inherent robustness and safety of the probe. Also, in view of the ability to easily shape the thin film as desired, an accurate probe can be produced after empirical testing and according to the desired functionality.

The deployable structure may comprise an inflatable or expandable balloon mounted at or proximate an insertion end of the catheter, some or all of the probes being mounted or formed on a surface of the balloon. In a variant, the deployable structure may comprise an expandable elastic basket structure or elastic beams mounted at or proximate an insertion end of the catheter, the probes being mounted or formed on a surfaces of or supported by the basket structure or elastic beams. The elastic basket or elastic beams may comprise a metal spring alloy per se known and used in invasive medical devices. The design of the expandable structure may be based on known expandable structures for medical applications, for instance similar to certain conventional devices for arterial stent placement.

The anemometric thin-film probes may be placed on an axis of the catheter, around the circumference and in other spatial position in order to establish a mapping of velocity components such as radial and angular velocity in a cylindrical geometry and to determine other hemodynamic parameters such as flow rate, pressure gradient, shear stress, and velocity moment.

The plurality of spatially arranged probes of the system according to the invention enables obtaining not only velocity vectors/components but also vorticity and turbulence characterizations inside blood vessels, thus providing extensive information and diagnostics on vessel and blood flow behavior and integrity.

The hot thin-film probes are particularly advantageous for measuring liquid flow, including turbulent flow or any flow in which rapid velocity fluctuations are of interest. Also advantageously, the thin-film probes are very compact and have extremely high frequency-response and fine spatial resolution compared to other measurement methods.

The system according to the invention thus enables provision of a more accurate and cost effective diagnosis than the one using known techniques such as angiography with pressure-wire guide or IVUS system. A major drawback of known systems is that they do not measure multiple flow velocity components at a cross section of an artery and thus do not provide accurate information on the morphology and functionality of the artery at that cross section. Conventional systems provide single flow velocity measurements at any cross section and thus only provide an approximate image of the average flow, without any characterization of either flow direction nor distribution.

In an embodiment, at least one probe of the sensor system may be chemically functionalized for use as an electric biosensor based for instance on an amperometric, potentiometric, or piezo-electric measurement in order to characterize not only blood but also plaques or specific biochemical coherent structures in arterial trees. Biochemical coherent structures may be defined as hemodynamic objects interacting with an internal artery wall where specific correlated flow pattern, mechanotransduction mechanisms between cells and specific biochemical (eg protein-protein) interactions take place as coherent events localized in space and in time, such factors being vectors for atherosclerosis.

In an embodiment, the probes may be embedded in controllable deployable elements such as flexible wires, basket, or balloon allowing to explore various positions and to measure the real flow over the artery cross section. This enables to deliver local relevant physiologic indices and identify mechanisms involved in the interaction of blood flow and the internal wall of arteries.

In an embodiment, the sensor system may further comprise at least one ultrasound transducer, for instance in the form of a piezoelectric transducer. The ultrasound transducer may be used to provide in vivo calibration of the anemometric probes by providing a measurement of the real volumic flow rate and the real time diameter of the artery. In a variant comprising a plurality of ultrasonic transducers, the characterization of coherent structures, such as correlated—in space and in time-vorticities may be performed.

In an embodiment, the catheter based system may comprise a deployable capsule or a patch mounted on the catheter and comprising therapeutically active agents or drugs for local therapy. The active agents may be administered to a relevant part of internal artery wall by taking advantage of local hemodynamic flow pattern information provided by the anemometric probes.

The system of the present invention not only proposes a functional and morphological cost effective diagnosis for interventional cardiologists but may also provide information on hemodynamic and vascular biology mechanisms in order to identify relevant biomarkers involved in vascular diseases such as vulnerable plaque.

A method according to an embodiment of the invention, includes diagnostic testing of atheriosclerosis by hemodynamic flow field measurements, including flow turbulence characterizations (vorticity parameters) and/or flow intermittency characterizations (velocity moment) and by morphological characterizations of the artery based on ultrasound piezo-electric transducers mounted on the catheter.

In an embodiment of the invention, the diagnostic testing of atherosclerosis by evanescent field techniques such as optical waveguide spectroscopy or surface Plasmon resonance technique may also be included. Testing may advantageously be based on a direct measurement of molecules and/or aggregates and/or markers interacting with at least one functionalized area linked to a catheter.

In an embodiment of the present invention, a multimodal catheter may advantageously regroup all or a subset of the above mentioned testing means.

Further objects, features and advantages of the present invention will be apparent to those skilled in the art upon a reading of this specification, the claims and the accompanying drawings.

### BRIEF DESCRIPTION OF THE DRAWINGS

FIG. 1a is a schematic illustration of a portion of catheter with anemometric hot-film sensors according to an embodiment of the invention; FIGS. 1b and 1c are cross-sectional views through lines 1b-1b and 1c-1c of FIG. 1a;

FIG. 2 is a schematic illustration of the behavior of a radial velocity component in an artery in a zone of stenosis;

FIG. 3 is a schematic illustration of a catheter based system according to an embodiment of the invention with expandable structure integrating probes such as thin-film probes and/or piezo-electric transducers;

FIG. 4 is a schematic illustration of a portion of catheter with anemometric piezo-electric ultrasound sensors according to an embodiment of the invention;

FIG. 5a, 5b are schematic cross-sectional illustrations of variants of a catheter with anemometric hot-film sensors according to an embodiment of the invention;

FIG. 6 is a schematic longitudinal section illustration of a portion of catheter with anemometric sensors on a deployable structure according to an embodiment of the invention;

FIG. 7 is a schematic perspective illustration of a portion of catheter with anemometric sensors according to an embodiment of the invention.

### DESCRIPTION OF EMBODIMENTS OF THE INVENTION

Referring to the figures, a catheter based system for providing functional and morphological characterization of arteries, comprising a catheter 1 configured for insertion in an artery 3, and a sensor system 5 for mapping hemodynamic parameters mounted on the catheter 1, the sensor system comprising at least two anemometric probes 7, 8a, 8b, 9a, 9b, 19, 20, 21, 22 spatially arranged in a deployed position and configured to measure flow velocity components (Vx, Vr) in at least two different positions spaced apart in a direction orthogonal to the axial direction (i.e. a radial direction R) of the catheter essentially in a same plane or within a short axial distance Dx or zone of the artery such that a possible restriction of the artery due for example to a stenosis, plaque, or other local deformation 3a of the artery is measurable. The short axial distance Dx thus corresponds to a distance encompassing a possible local artery restriction 3a, typically in the order of less than two or three times the average diameter Dr of the artery to be mapped,

The anemometric probes may advantageously comprise hot thin-film probes. The use of hot thin-film probes is particularly advantageous in view of the easy and flexible forming thereof in a desired configuration on a deployable structure or on the surface of the catheter, and the inherent robustness and safety of the probe. Also, in view of the ability to easily shape the thin film as desired, an accurate probe can be produced after empirical testing and according to the desired functionality.

Instead of hot thin-film probes, the anemometric probes may nevertheless also include probes in the form of hot wire probes or anemometric probes of other types or configurations.

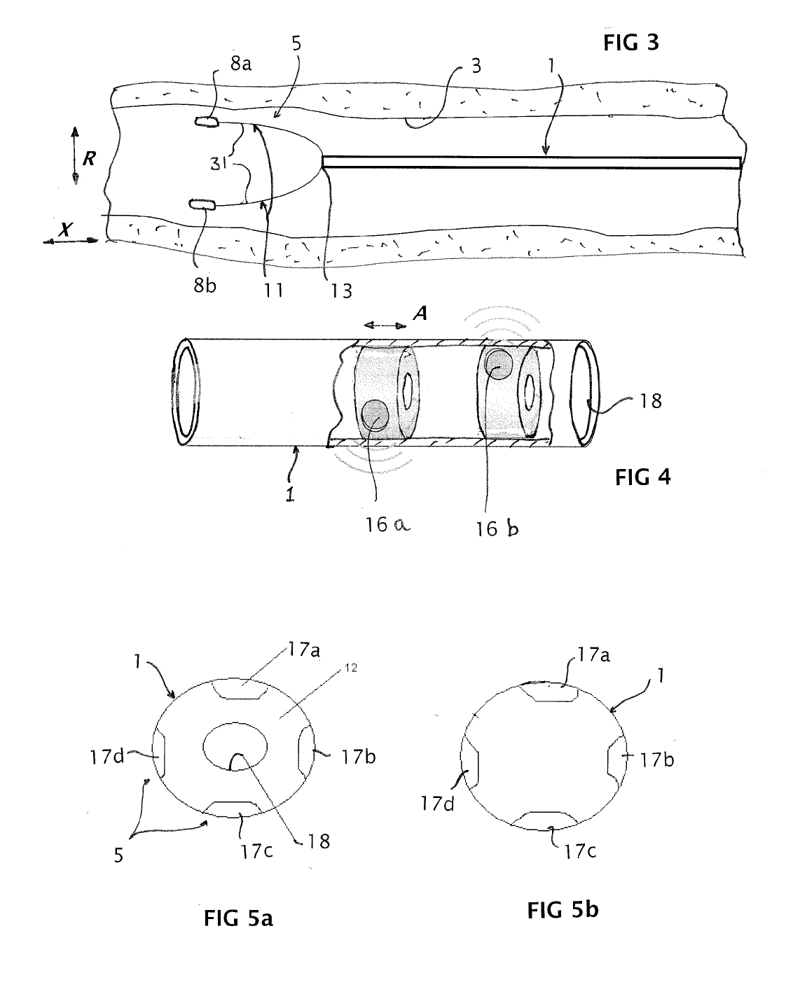

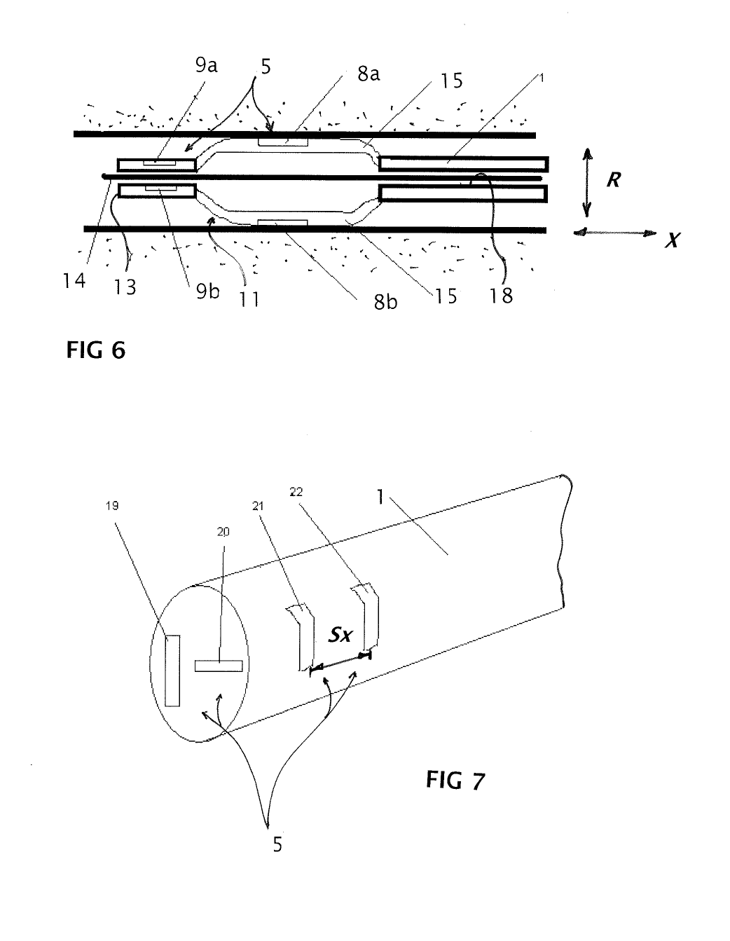

At least one of the anemometric probes, but preferably at least two, may advantageously be mounted on a deployable structure 11 configured to expand in at least a radial direction R from a retracted position allowing insertion of the catheter in an artery 3, to the deployed position as illustrated in FIGS. 3 and 6 for mapping of hemodynamic parameters.

The deployable structure may comprise an inflatable or expandable balloon mounted at or proximate an insertion end 13 of the catheter 1, the probes 8a, 8b, being mounted or formed on a surface of the balloon. In a variant, the deployable structure may comprise an expandable elastic basket structure 15 or elastic beams 31 mounted at or proximate an insertion end 13 of the catheter, the probes being mounted or formed on a surfaces of or supported by the basket structure or elastic beams. The elastic basket or elastic beams may comprise a metal spring alloy per se known and used in invasive medical devices. The design of the expandable structure may be based on known expandable structures for medical applications, for instance similar to certain conventional devices for arterial stent placement.

Anemometric probes 9a, 9b, 19, 20 may be placed on or proximate an axial axis of the catheter, and/or around the circumference 7, 17a,17b,17c,17d, 21, 22 and in other spatial positions in order to establish a mapping of velocity components such as radial Vr and angular Vx velocity in a cylindrical geometry and to determine other hemodynamic parameters such as flow rate, pressure gradient, shear stress, and velocity moment.

The catheter 1 may comprise a lumen 18, or a plurality of independent lumens (not shown) which may be configured for one or more different functions, including receiving a guide wire 14 for insertion of the catheter and/or for remote control of deployment of the expandable deployable structure 15, a conduit for hydraulic or gaseous expansion of an inflatable balloon structure, a conduit for electrical leads, and/or a conduit for the local delivery of a therapeutic agent in the measurement zone.

The sensor system 5 of the catheter based system may advantageously further include at least one probe in the form of an ultrasound transducer 16a, 16b mounted in the catheter 1 or on the surface of the catheter configured to provide a measurement of volumic flow rate and/or morphological information on artery morphology. The ultrasound transducer transmits an ultrasound signal that is reflected back from the artery wall and received by the transducer, enabling measurement of the distance and thus a determination of the local diameter. Also, based on the Doppler effect, the transducer may also be used to compute average blood flow velocity, which may then be used to calibrate in situ and in vivo the anemometric hot thin film or hot wire sensors. The ultrasound transducer may for instance be a piezoelectric transducer.

Instead of an ultrasound transducer, in a variant the sensor system may comprise an electromagnetic or capacitive sensor configured to provide a measurement of volumic flow rate and/or morphological information on artery morphology.

The sensor system 5 of the catheter based system may advantageously further include at least one probe that may be chemically functionalized for use as an electric biosensor, the probe being mounted either on the catheter or on the expandable structure 11, 15.

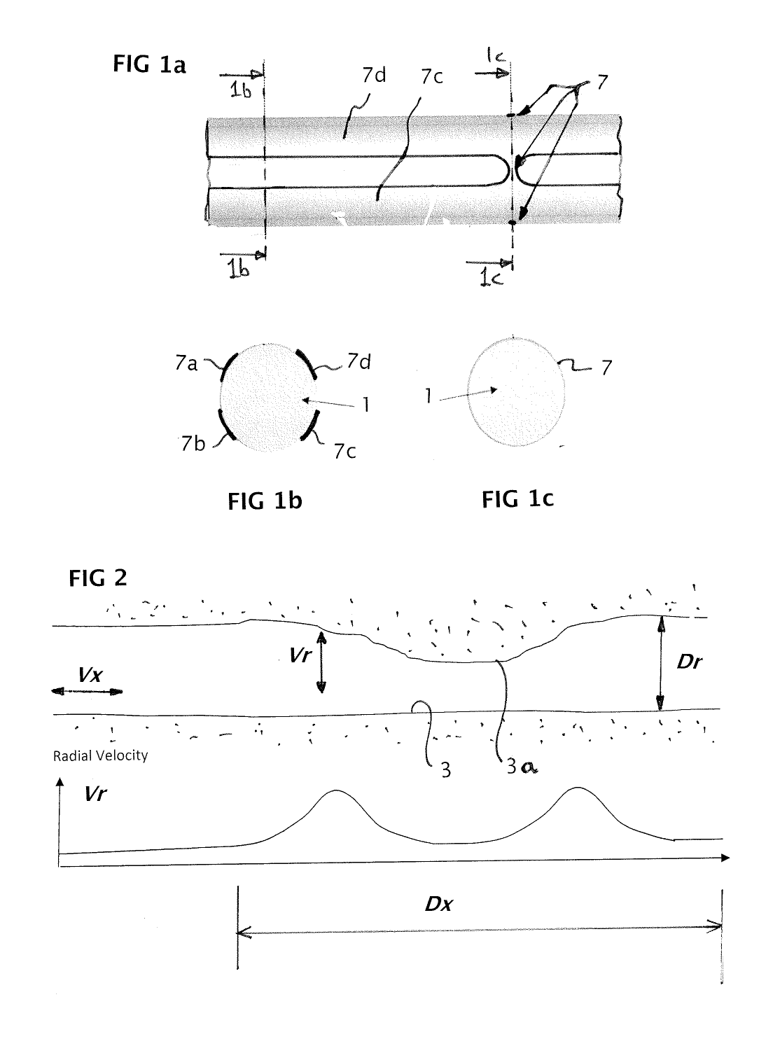

In FIG. 1a-1c the portion of catheter comprises a thin-film anemometric arrangement that is shaped on the surface of the cylindrical catheter. In this example, four independent hot spots 7 are arranged around the catheter substrate or outer surface essentially in a same radial cross-sectional plane 1c-1c. It can be envisaged to have more than four hot spots for better resolution or to arrange them spaced differently, fro instance some offset with respect to the plane 1c-1c. An important condition is that the conductivity of the contacts 7a-7d is much higher as the conductivity of the hot spots 7;

FIG. 4 illustrates a portion of catheter 1 with a plurality of ultrasonic emitter-receiver transducers 16a, 16b. The transducers are in this example in the form of longitudinal polarized piezo-rings arranged behind windows in the catheter. The double-arrow A indicates oscillation due to external polarization, whereby an acoustic wave propagates through the window. The catheter itself plays the role as fixation and damping element. Moreover, it could further integrate a plurality of thin-film probes on the catheter surface or on an expandable deployable structure to perform hemodynamic sensing function.

FIG. 6 illustrates an embodiment of the invention where the diagnosis of an internal artery wall 3 is performed with a catheter 1 comprising an expandable structure 15. The probes 8a, 8b on the expandable structure may include chemically functionalized probes (i.e. biosensors) in contact with the artery wall. The biosensor probes may comprise dissipative quartz microbalances allowing first to obtain quantitative viscoeleastic properties of the artery (and detection of wall contact) and, second, thanks to its chemically functionalized surface, to characterize the biochemical species present on the wall. Each biosensor probe may be protected by a layer in order that its chemically functionalized part is not damaged before the convenient position on the artery wall. This protective layer can be removed mechanically (such as film tube) or by physical processing (local heating for its dissolution). In order to perform in one run functional and morphological diagnosis, the anemometric thin film probes and optionally in addition ultrasound probes are integrated in the catheter on the expandable structure 15 at positions referenced 8a, 8b where the probe is not a biosensor or on the catheter at positions referenced 9a, 9b. The guidewire 14 in the lumen 18 may be used to provide mechanical support to introduce expand the structure 15.

In FIG. 7, a configuration of probes 19, 20, 21, 22, which may include hot thin-film probes, are integrated on the catheter 2 for functional diagnosis, the thin-film probes having typical size around hundreds to tens of microns acting as anemometers to measure blood velocity components and their moments (of order 2, order 3, or order 4) or velocity correlation functions.

The configuration of certain anemometric thin-film probes 21, 22, axially spaced apart enables the measurement of shear stress through the calculation of space velocity gradient; the resolution is given by the axial space Sx between probes 21 and 22. Moreover, pulse anemometry technique may advantageously be applied between probes 21 and 22, to estimate potential backflow of the blood.

The catheter body or a part of the catheter body may be configured as an optical waveguide, the probes at positions similar to 21, 24 on the surface of the optical wave guide comprising a chemically functionalized film on which optical evanescent techniques may be applied. The probes 21, 24 may also be biosensors comprising chemically functional thin film functioning on amperometric or potentiometric principles. The biosensors on the catheter surface may provide biochemical information on blood flow inside artery.

A feature of the present invention is a method using thin-film anemometry methodologies applied for hemodynamic characterizations. One advantage of the present invention is the capabilities to detect and measure local hemodynamic observables such as mean blood velocity and its temporal fluctuations. Better understanding is then available regarding mechano-transduction processing between blood flow and the very first cellular layers from vessel walls. The size of the thin-film probe sensing area gives the spatial resolution of the measurements.

A feature of the present invention includes a catheter comprising at least two thin-film probes for the measurements of blood velocity components and/or velocity moments and/or vorticity. According to specific probe configuration (thin-film probe in X shape for example) the device of the present invention can evaluate the two dimensional velocity components. From these measurements, several observables are available: for example, according to the relation Vorticity˜Rotational (velocity), the vorticity of blood flow can be quantified and characterizing the turbulence of blood flow.

In an embodiment of the present invention is that the thin-film probes allow the measurements of further physical quantities such as the temperature and/or the strain field especially in the intravascular boundary layer.

In order to perform such measurements without the perturbations generated by the presence of the catheter body at least one probe is mounted on a moveable element respective to the catheter body like a filament or a ballon. The latter could be expandable from an operation with the catheter body. Electrical means are integrated into the signal to detect and transmit signal.

In an embodiment at least one of the probes is chemically functionalized and then can be used as biosensors in order to characterize not only blood but also plaques or specific coherent structures in arterial tree. The main advantage is to provide a—cost effective—functional and morphological diagnosis of arteries.

In an embodiment the probes are embedded in fine folding elements (for example such as flexible wires) allowing to explore various position and to measure the real flow. The advantage is to deliver local relevant physiologic indices and identifiy mechanisms involved in the interaction of blood flow and the internal wall of arteries.

In an embodiment the probes are coupled with at least one piezoelectric transducer. The advantages are the following: in vivo calibration of probes and measurement of the real volumic flow rate. In the case of the integration of a plurality of ultrasonic transducers, the advantage is the characterization of coherent structures, through for example the vorticity.

The piezo-electric transducer can be designed with annular geometry (FIG. V) or piezoelectric coated materials

An embodiment of the present invention is that the device integrates at least two pairs of piezoelectric transducers comprising two emitting transducers and two receiving transducers such that between said emitters and said receivers two angles are defined performing acoustic spectroscopy and platelet characterizations or acoustic interferometry which allows the direct and global (average on a finite volume of the flow) probing of the spatio-temporal dynamics of the vorticity field in turbulent flow.

In an embodiment, the device is integrated such that it can deliver active therapeutic agents (drugs) for local therapy by deploying a patch incorporating active agents adherent to a relevant part of internal wall artery or by supplying liquid drug(s) taking advantage of local hemodynamic pattern.

In an embodiment of the present invention, evanescent field technique and associated probe(s) embedded in a catheter is employed to perform in vivo biochemical analysis and diagnosis. The required optical waveguide(s) is integrated into the catheter. Preferentially it is moveable with respect to the catheter body, and could be associated with an expandable configuration, like a basket-shape or egg-shape catheter.

The principle of the technique in an embodiment of the present invention is the following: at least one light source is guided and interacts on at least one grating or at least one specific microstructured optical element at an interface with relevant intravascular parts up to at least one detector. The interface between the external surface of the grating or the microstructured element with the intravascular parts could be functionalized. Sensitive from refractive index changes within the evanescent field. The thickness and refractive index of the adsorbed layer at the interface can be measured, the mass too. Relevant molecules from vessel walls or relevant interactions with marker(s) can be estimated. In order to measure at a specific position onto the vessel wall, a protective layer of the interface is desirable.

An embodiment of the present invention may include a method employing surface plasmon resonance probe(s) embedded in a catheter to perform in vivo biochemical analysis and/or diagnosis. The said probe comprises a thin metallic coating for example mounted on a prism. Light passes through the prism, reflects off the metallic coating and passes back through the prism to a detector. Changes in reflectivity versus angle or wavelength give a signal that is proportional to the volume of biopolymer bound near the surface. The surface plasmoms are electromagnetic waves excited by light in such meta films. This principle requires protecting the sensing area(s) before the correct positioning of the catheter in the vessel, thanks to moveable shield(s). An alternative is to protect the sensing area thanks to at least one material layer (solid, or gel-like) and to remove physically and/or chemically the said layer. By monitoring the signal from this dissociation, it is possible to end point detect the complete removing of the protective layer and start the measurements (internal vessel walls).

In order to enhance the sensitivity levels, it is advantageous to employ surface nanopatterning techniques at the metallic interface by increasing the said angle.

An embodiment of the present invention may include the multimodal physical and chemical intravascular characterizations and/or diagnostics by regrouping the said thin-film anemometry methodologies and/or the said quartz crystal microbalance technique (including operation in pulse mode to provide information about the viscoelastic properties—i.e., shear modulus, anisotropy, viscosity . . . —of relevant part of internal wall of artery, such as plaque) and/or acoustic spectroscopy method in a unique catheter.

The catheter-like device comprises sensing elements with transducers and/or sensors based on ultrasound and/or optical and/or electromagnetic principles.

An embodiment of the invention may include a catheter having a basket-shaped element array with a plurality location sensors (such as thin-film probe and/or piezoelectric transducer) mounted at its distal end. The catheter may comprise an elongated catheter body having proximal and distal ends and a basket-shaped element assembly mounted at the distal end of the catheter body.

The catheter body comprises an elongated tubular construction that may have a single, axial or central lumen, but can optionally have multiple lumens if desired. The catheter body is flexible, i.e., bendable, but substantially non-compressible along its length. The outer wall may comprise an imbedded braided mesh of stainless steel or the like to increase torsional stiffness of the catheter body so that, when the control handle is rotated, the distal end of the catheter body will rotate in a corresponding manner. The thickness of the outer wall is preferably thin enough so that the central lumen can accommodate a puller wire, lead wires, sensor cables and any other wires, cables or tubes. The elements are all attached, directly or indirectly, to the expander at their distal ends, and to the catheter body at their proximal ends. The expander is moved longitudinally to expand and contract the element assembly, so that, in the expanded position the elements are bowed outwardly and in the contracted position the elements are generally straight. As will be recognized by one skilled in the art, the number of elements can vary as desired depending on the particular application. As used herein, the term “basket-shaped” in describing the element assembly is not limited to the depicted configuration, but can include other designs, such as spherical or egg-shaped designs, or a series of elementary design, that include a plurality of expandable arms connected, directly or indirectly, at their proximal and/or distal ends.

In an embodiment, said element is made of flexible, biocompatible materials, with non-conductive coating.

If desired, the catheter can include a steering mechanism for deflection of the distal end of the catheter body. Preferably the handle has a pair of movable members to which the expander and puller wire attach, such as handles typically used for bidirectional and multidirectional catheters.

In an embodiment of the present invention, said element and/or the catheter body and or the probes and/or the sensing areas are microstructured for specific functionalities such as hydrophilic function to better progress into vascular network, cell aggregate capturing for biochemical analysis.

In an embodiment of the present invention is that the device uses pulsed thin-film probes for spatial correlation and/or reverse flow measurements: flow velocity is deduced from the time taken for the thermal wake of a thin-film probe, heated by a short pulse of current, to reach a—sensor—thin-film probe operating as a resistance thermometer.

The catheter can be a guide wire type with a diameter around 0.3 mm to 3 mm, for instance in a range of 0.5-1.5 mm, whereby a catheter with guide wire would be normally in a range of 1 mm to 3 mm diameter.

Advantageous features of the invention may include:

1. Catheter based Hot-film anemometry device and method applied for blood flow characterizations or intravascular flow characterizations such that the components of blood flow velocity vector is measurable, including at least one velocity component in a Cartesian reference or in a cylindrical reference.

2—In addition to the above feature, further measuring the blood flow vorticity.

3—In addition to the above features, further measuring the blood flow velocity moments.

4—In addition to any one of the previous features, further measuring the strain field inside the vessel.

5—A method and device for blood flow characterizations comprising: providing at least one thin-film probe linked to a catheter body having proximal and distal ends, said thin-film electrically heated up and configured to be cooled by blood flowing past the thin-film whereby the electrical resistance of said thin-film is dependent upon the temperature of the thin-film, and determining blood flow characteristics based on a predetermined relationship between the resistance of the said thin-film and the flow velocity.

6—In addition to the feature 5 above, providing at least two thin-film probes arranged to form an X shape allowing the measurement of the two-dimensional blood flow velocity components.

7—In addition to the feature 5 above, providing three thin-film probes configured to provide three-dimensional information, said thin-film probes optionally aligned with axes of an orthogonal system of co-ordinates to allow the simultaneous determination of the three blood flow velocity components (Vx, Vy, Vz) in a Cartesian reference.

8—In addition to the feature 5 above, providing a plurality of thin-film probes such that the said probes are arranged according the circumference of the catheter allowing the measurement of the angular velocity components.

9—In addition to the feature 8 above, providing further thin-film probes according to the longitudinal axis of the catheter to allow radial velocity components.

10—In addition to the feature 5 above, further measuring blood flow strain field.

11—Catheter based anemometry including at least one evanescent field sensor employing evanescent light technique and comprising at least one optical waveguide, at least one optical mean and at least one chemically and/or physically functionalized area such that specific molecule(s) and/or marker(s) can be identified, said sensor being linked to a catheter body having proximal and distal ends.

12—In addition to the feature 11 above, the evanescent field sensor further comprising at least one metallic layer deposited on a part of the said optical waveguide and a part of the said optical mean such that surface Plasmon resonance analysis are performed by measuring a resonant coupling between the light and the surface plasmons in the said metallic layer occurs at a specific angle, whereby reflected light produces a “shadow” at the resonance angle, said angle being sensitive to the adsorption of relevant molecules and/or markers at the interface and wherein said optical mean optionally has a prism function.

13—In addition to the feature 11 above, the optical means may comprise at least one grating or at least one microstructured part and performs optical waveguide spectroscopy by measuring the thickness and/or the refractive index of adsorbed relevant molecules.

14—In addition to the feature 11 above, providing at least one protective layer dedicated to the said evanescent light sensor such that the said protective layer can be removed physically or chemically in order to perform measurements with the said evanescent light sensor by exposing the said functionalized area.

15—In addition to the feature 11 above, providing at least one of the said evanescent field sensor mounted in an expandable element assembly linked to the said catheter body.

16—A method and device for intravascular diagnosis comprising means for performing a combination of hot-wire anemometry method and surface Plasmon resonance method such that the intravascular blood flow and the vessel walls and aggregates as vulnerable plaques can be substantially fully characterized for clinical diagnostic.

17—A method and device for intravascular diagnosis comprising means for performing a combination of hot-wire anemometry method and optical waveguide spectroscopy method such that the intravascular blood flow and the vessel walls and aggregates as vulnerable plaques can be substantially fully characterized for clinical diagnostic.

18—A catheter based system wherein one or some of the probes are chemically functionalized and then can be used as biosensors to measure the concentration of at least one molecule or marker; and means for transmitting to the proximal end thereof the said measurement received from the said probe.

19—In addition to feature 18 above, the catheter may further comprise at least one piezoelectric ultrasound transducer allowing in vivo calibration of said probes and measurement of the volumic flow rate.

20—In addition to feature 19 above, there may be at least two pairs of piezoelectric transducers comprising two emitting transducers and two receiving transducers such that between said emitters and said receivers two angles are defined performing acoustic spectroscopy and platelet characterizations or acoustic interferometry

21—The catheter may further comprise means to deliver active agents (drugs) for local therapy.

22—The probes and/or said piezoelectric transducers may be embedded in fine folding elements (for example such as flexible wires or flexible optical fibers) allowing to explore various position and to perform functional and morphological (for example vascular remodeling) measurements.

23—The piezoelectric transducer may be used to perform quartz crystal microbalance function including operation in pulse mode in order to acquire information on viscoelastic properties and anisotropy of plaque and surrounding environment or molecule or marker identification.

24—The catheter based system may comprise at least one of said functionalized area mounted or coated on an expandable element assembly linked to the catheter.

25—The expandable elements may be configured to bow radially outwardly when the assembly is in the expanded or deployed position.

26—The catheter may be sized and configured for interrogation of human arteries or aneurysm diagnosis or heart valve performances.

1-16. (canceled)

17. Catheter based system for providing functional and morphological characterization of arteries, comprising a catheter configured for insertion in an artery, and a sensor system mounted on the catheter, wherein the sensor system includes a plurality of probes comprising at least two anemometric probes spatially arranged in a deployed position and configured to measure flow velocity components in at least two different positions spaced apart in a direction orthogonal to the axial direction of the catheter.

18. Catheter based system according to claim 17, wherein the anemometric probes comprise hot thin-film probes.

19. Catheter based system according to claim 17, wherein at least one of the at least two anemometric probes is mounted on a deployable structure configured to expand from a retracted position allowing insertion of the catheter in an artery, to the deployed position.

20. Catheter based system according to claim 19, wherein the deployable structure comprises an inflatable or expandable balloon mounted at or proximate an insertion end of the catheter, some or all of said plurality of probes being mounted or formed on a surface of the balloon.

21. Catheter based system according to claim 19, wherein the deployable structure comprises an expandable elastic basket structure or elastic beams mounted at or proximate an insertion end of the catheter, some or all of said plurality of probes being mounted or formed on a surfaces of or supported by the basket structure or elastic beams.

22. Catheter based system according to claim 17, wherein at least one probe may be chemically functionalized for use as an electric biosensor.

23. Catheter based system according to claim 22, wherein at least one of said at least on chemically functionalized probe is mounted on a deployable structure configured to expand from a retracted position allowing insertion of the catheter in an artery, to the deployed position.

24. Catheter based system according to claim 17, wherein at said plurality of probes comprises at least one ultrasound transducer configured to provide a measurement of volumic flow rate and/or morphological information on artery morphology.

25. Catheter based system according to claim 24, wherein the ultrasound transducer is a piezoelectric transducer.

26. Catheter based system according to claim 24, wherein there are a plurality of ultrasound transducers.

27. Catheter based system according to claim 24, wherein the ultrasound transducer is mounted in the catheter.

28. Catheter based system according to claim 17, wherein the catheter comprises a lumen with a guide wire inserted therein for actuating deployment of an expandable structure.

29. Catheter based system according to claim 17, wherein at least one of said plurality of probes is mounted on an outer surface of the catheter.

30. Catheter based system according to claim 17, wherein at least two of said plurality of probes is mounted on an outer surface of the catheter.

31. Catheter based system according to claim 17, wherein said plurality of probes comprises at least two anemometric probes spatially arranged in an axial direction and configured to measure flow shear stress and/or backflow.

32. Catheter based system according to claim 17, further comprising a deployable capsule or a patch mounted on the catheter comprising active agents or drugs for local therapy, or a conduit in the catheter configured for transporting therapeutically active agents or drugs for local therapy.
